(19) United States
(12) Patent Application Publication
(10) Pub. No.: US 2002/0015971 A1
(43) Pub. Date: Feb. 7, 2002

(54) METHOD FOR DETECTING RARE EVENTS

(76) Inventor: BEN J. H. VERWER, SANTA JOSE, CA (US)

Correspondence Address: RICHARD J RODRICK, BECTON DICKINSON AND COMPANY, 1 BECTON DRIVE, FRANKLIN LAKES, NJ, 074171880

(21) Appl. No.: 09/048,222

(22) Filed: Mar. 26, 1998

Related U.S. Application Data
(63) Continuation-in-part of application No. 08/743,563, filed on Nov. 4, 1996, now abandoned.
Application No. 08/743,563 is a continuation of application No. 08/318,488, filed on Oct. 5, 1994, now abandoned.
Application No. 08/318,488 is a continuation of application No. 08/009,185, filed on Jan. 26, 1993, now abandoned.

(51) Int. Cl. G01N 33/53

(52) U.S. Cl. 435/7.24

(57) ABSTRACT

This invention relates to a method for the detection of rare events which are events occurring at a frequency of less than one in 10⁴. It is particularly useful in detecting events at frequencies of one in 10⁵ and one in 10⁶. The method employs one or more first markers that are specific for the rare event particle each of which are labeled with a dye having an emission wavelength distinguishable from the other(s). The method further employs one or more second markers that are specific for a majority of the remainder of the particles present in the cell sample but are negative for the rare event particle. The second markers all are labeled with the same dye. The second markers are collectively referred to as the exclusion color. By analyzing the particles for the presence of the first marker(s) and the absence of the second markers, rare events can be detected.

This is a continuation-in-part of U.S. patent application Ser. No. 08/743,563, filed Nov. 4, 1996, which is a continuation of U.S. patent application Ser. No. 08/318,488, filed Oct. 5, 1994, now abandoned, which is a continuation of U.S. Pat. application Ser. No. 08/009,185, filed Jan. 26, 1993, now abandoned.

### FIELD OF THE INVENTION

This invention relates to a method for the detection of rare events in a sample of particles, and more particularly relates to a method for the detection of rare cells in a sample of cells at frequencies of less than one in 10⁶ cells.

### BACKGROUND OF THE INVENTION

Particle analysis generally comprises the analysis of cells, nuclei, chromosomes and other particles for the purpose of identifying the particles as members of different populations and/or sorting the particles into different populations. This type of analysis includes automated analysis by means such as flow cytometry. In flow cytometry, the particle, such as a cell, may be labeled with one or more markers and then examined for the presence or absence of such markers. In the case of a cell, such as a leukocyte, tumor cell or microorganism, the marker can be directed to molecules on the cell surface, such as receptor proteins, or to molecules in the cytoplasm or nucleus such as proteins, enzymes or nucleic acids. Examination of a particle's physical characteristics (e.g., size), as well as for the presence or absence of marker(s) can provide additional information which can be useful in identifying the population to which a particle belongs.

Flow cytometry comprises a well known methodology using multi-parameter data for identifying and distinguishing between different particle types in a sample. For example, a sample of cellular particles may be drawn from a variety of biological fluids such as blood, lymph, bone marrow, ascites, pericardial fluid, pleural fluid, cerebrospinal fluid or urine, or may be derived from suspensions or aspirates of cells from organs and solid tissues such as brain, GI tract, prostate, lung, breast, kidney, lymph node or liver. In a flow cytometer, cells are passed substantially one at a time through one or more sensing regions where in each region each cell is illuminated by an energy source. The energy source generally comprises an illumination means that emits light of a single wavelength such as that provided by a laser (e.g. He/Ne or argon ion) or a mercury arc lamp with appropriate bandpass filters. Light at 488 nm is a presently preferred wavelength of excitation when a single light source is used and 632 nm is preferred as a second light source when two light sources are used.

In series with one or more sensing regions, multiple light collection means, such as photomultiplier tubes, are used to record light that passes through each celi (generally referred to as forward light scatter), light that is reflected orthogonal to the direction of flow of the cells through the sensing region (generally referred to as orthogonal or side light scatter) and fluorescent light that may be emitted from the cell, if it is labeled with fluorescent marker(s), as it passes through the sensing region and is illuminated by the energy source. Each of forward light scatter (or FSC), orthogonal light scater (SSC), and fluorescence emissions (FL1, FL2, etc.) comprise a separate parameter for each cell (or each “event”). Thus, for example, up to four parameters can be collected (and recorded) from a cell labeled with two different fluorescence markers (Le., two scatter parameters and two fluorescent parameters per cell).

Flow cytometers further comprise data acquisition, analysis and storage means such as a computer equipped with appropriate software. Signals from each PMT are converted into multiple data channels and stored by the computer. The purpose of the analysis system is to classify and count cells wherein each cell presents itself as a set of digitized parameter values. Typically, by current analysis methods, data collected in real time (or recorded for later analysis) is plotted in 2-D space for ease of visualization.

Such plots are referred to as dot plots and a typical example of a dot plot drawn from light scatter data recorded for leukocytes is shown in FIG. 1 of U.S. Pat. No. 4,987,086.

By plotting orthogonal light scatter versus forward light scatter, one can distinguish between granulocytes, monocytes and lymphocytes in a population of leukocytes isolated from whole blood. By electronically (or manually) gating on only lymphocytes using light scatter, for example, and by the use of the appropriate monoclonal antibodies labeled with fluorochromes of different emission wavelength, one can further distinguish between cell types within the lymphocyte population (e.g., between T helper cells and T cytotoxic cells). U.S. Pat. Nos. 4,727,020, 4,704,891, 4,599,307 and 4,987,086 describe the arrangement of the various components that comprise a flow cytometer, the general principles of use and one approach to gating on cells in order to discriminate between populations.

While the above-described methods are useful and accurate for the analysis of particles that occur in a sample at a frequency of greater than 1%, accuracy is much less reliable in the analysis of particles that occur with lesser frequency. These events are termed “rare events.”

Accuracy in the counting and analysis of rare events is important. For example, in many cancers elimination of “cancer cells” is the desired goal of a therapy. It is important to know if all of the cancer cells have been eliminated. If a number of cancer cells remain but are undetected because existing instrumentation cannot reliably detect the cells that remain because of their low frequency, a patient could be given an erroneous “clean bill of health.” Similarly, if these minimum residual cells have not been eliminated, it is important for monitoring purposes to detect increases in the frequency of such cells at the earliest possible time in order to determine whether changes in treatment are required. Thus, accurately quantitating the number of cancer cells that remain after treatment is important in the future treatment and monitoring of cancer.

A concern in the accurate counting of cells by flow cytometry is assuring that an event which the instrument senses as being positive for a “fluorescent” marker actually is so as a result of being labeled with a fluorescent marker. It has been reported that freshly isolated unlabelled mouse bone marrow contains autofluorescent particles that a cytometer senses as being “fluorescent.” The incidence of these events was reported as one per 10⁴ cells. As a result, it has been considered that the lowest achievable level of detection using commercially available cytometers is about one cell in 10⁵.

In addition to unlabeled cells being recorded at some frequency as “fluorescent” events, the use of immunofluorescence markers can cause additional errors in data analysis. Monoclonal antibodies labeled with fluorescent dyes have a high affinity for their antigen. Unfortunately, because of the nature and structure of the cells on which such antigens appear, immunofluorescence markers will bind to structures other than the intended antigen. Thus, irrelevant cells can be recorded as positive fluorescent events due to non-specific labeling. For cells whose frequency is below 1% of the cells in a sample, this is an additional problem to be addressed.

A third problem with conventional cytometry is the speed at which cells are analyzed. Current instrumentation operates at an acquisition rate of 5,000 to 10,000 events per second. Current instrumentation also has a limited random access memory associated with the data analysis hardware to acquire events for real time analysis. Thus, to acquire about 108 events, it takes about 3 to 6 hours at a minimum. To maintain cells over such a long period of time with the same fluorescent characteristics, it is necessary to fix the cells. Fixation will have some effect on the physical characteristics of the cell as opposed to fresh living cells, and thus, presents a further opportunity for the introduction of error into the system.

Accordingly, an improved method for the analysis of cells is required if rare event analysis is to be a more useful and accurate tool in cytometry.

### SUMMARY OF THE INVENTION

This invention comprises a high-speed method for the detection of particles in a sample at frequencies of less than one in 10⁴.

The particles can be cells in biological fluids, including red and white blood cells, dis-aggregated cells from organs and solid tissues and microorganisms. Samples of such cells can be taken from blood, urine, bone marrow, ascites, pericardinal fluid, pleural fluid, cerebrospinal fluid, lymph and other biological fluids, as well as from organs and solid tissues such as brain, kidney, prostate, liver, spleen, lung, breast, lymph node and GI tract.

The particles to be analyzed are combined with at least one first marker that is specific for (i.e., has a high binding affinity for) the particles to be detected and at least one second marker that is specific for the majority of the remainder of the particles in the sample but is negative for (i.e., has low or no binding affinity for) the particles to be detected. It should be appreciated that in few circumstances will there be a single marker which will be specific for the majority of remaining particles in a sample. Therefore, most uses will require more than one second marker. This second marker also can be referred to as the exclusion marker.

Where the particles comprise cells, it is preferred that the markers be immunofluorescence markers. Where more than one immunofluorescence marker is used, it is preferred that each of the first markers be labeled with a different fluorescent dye. Each such dye should have an emission wavelength that is distinguishable from the other(s). It further is preferred that the second marker be labeled with a fluorescent dye that has an emission wave length distinguishable from that of the dyes used in connection with the first marker(s). It should be noted, however, that while each of the first markers generally is labeled with a different dye (when multiple first markers are used), when more than one second marker is used they all preferably are labeled with the same dye. Finally, it is to be appreciated that where multiple markers are used they can comprise combinations of i-mmtinofliiorescence markers and other cytoplasmic or nuclear markers (e.g., nucleic acid dyes).

In the preferred embodiment, the number of first markers is two, and the number of second markers is greater than one.

This invention has particular utility in the detection of cancer cells in bone marrow or peripheral blood preparations of stem cells. Stem cells have been described as being CD34+. Subsets of such cells include cells that are CD34+/CD38−, cells that are CD34+/CD38−/HLA-DR+and cells that are CD34+/CD38−/HLA-DR−. In the treatment of patients requiring infusion of stem cells, it is important to eliminate any remaining cancer cells from the preparation. For those cancers that are CD34−, antibodies against CD34 would comprise the exclusion color while antibodies specific for the cancer cells would comprise the first marker(s).

### BRIEF DESCRIPTION OF THE DRAWINGS

FIG. 1 is a plot of natural log probability of each sequence of 1000 events for a sample of 1.40×10⁶ cells containing rare events at a frequency of about 10⁻⁶. Each of the probabilities is calculated using the relative frequencies of parameter values in accordance with the method set forth below.

FIGS. 2A, 2B and 2C are schematic diagrams of the gates applied to n-dimensional data (and shown in 2-D form) in order to isolate rare events. FIGS. 3A, 3B, 3C, 3D, 3E, 3F, 3G, 3H and 3I comprise a series of dot plots of (A)-(G) SSC versus FSC, (H) FL2 versus FL1 and (I) FL2 versus FL3 for various amounts of REII cells seeded into 10⁸ PBMC.

### DETAILED DESCRIPTION OF THE PREFERRED EMBODIMENTS

Peripheral blood monoculear cells (“PBMC”) were isolated from buffy coats by density gradient separation. After two washes with 0.45μ filtered Ca²⁺and Mg²⁺free phosphate buffered saline (“PBS”), the cells were resus pended in 0.45μ filtered PBS in 20% fetal calf serum (“FCS”). Cell suspensions of more than 1.2×109 cells were rotated for one hour. Viability was determined by the propidium-iodide exclusion test on a FACScan brand flow cytometer (Becton Dickinson Immunocytometry Systems, “BDIS”). Viability was always greater than 98%.

Cells not stained were counted on a hemocytometer. 2.5×108 PBMC were aliquoted in up to 4 separate Isml tubes. Each tube was supplemented 0.45μ filtered FCS to a total 10 volume of 14 ml.

The pre-B cell line REH was cultured in RPMI in 10% FCS in an atmosphere of 7% CO₂ at 37° C. Twelve hours prior to use, the cultures were split and fresh medium was added. The cells were harvested and washed with 0.45 μ filtered PBS prior to sort. Viability was determined as above. Viability was always greater than 94%.

Where required, pre-B cells were labeled with immunofluorescence markers as follows. Pre-B cells were sorted as described below into 2.5×10⁸ PBMC. The mixture then was rotated for one hour to ensure dispersion. The cells then were spun down and supernatant removed to leave about 50 μl of solution. 50 μl of normal mouse serum (Dako.) was added. The cells were resuspended and rotated for one hour.

190 μl of labeling cocktail was added. The cocktail contained 25% normal mouse serum and the following antibodies at the following final sample concentrations. To separate pre-B cells from mature B cells, anti-CD19 fluorescein isothiocianate (Leu 12 FITC, BDIS) 2.4 μg/,l were added as a first marker. To exclude T cells, granulocytes, monocytes and platelets, anti-CD7 peridinin chlorophyll protein (Leu 9 PerCp, BDIS) 22.9 μg/ml, anti-CD3 PerCP (Leu 4 PerCp, BDIS) 5.4 μg/ml, anti-CD4 PerCp (Leu 3a PerCp, BDIS) 1.6 μg/ml, anti-CDS PerCP (Leu 2a PerCP, BDIS) 2.0 μg/ml, anti-CD15 PerCP (Leu MI PerCp, BDIS) 4.7 μg/ml, anti-CD15 PerCp (L-16 PerCp) 6.4 μg/ml, anti-CD14 PerCp (Leu M3 PerCp, BDIS) 1.3 μg/ml, anti-CD41a PerCp 12.3 μg/ml and anti-CD42b PerCp 29.5 μg/ml were added as second markers. For additional identification of pre-B cells, anti-CD10 phycoerythrin (anti-CALLA PE, BDIS) 6.2 μq/ml was added, as an additional first marker. As an isotype control, anti-KLH PE 6.2 μg/ml was added.

The cells then were resuspended and rotated for one hour to ensure complete labeling. The cells were washed twice in PBS and fixed in a total volume of 200 ml in 0.5%, paraformaldehyde for 12 hours. After harvesting, the cells were washed once in PBS and finally resuspended in a total volume of 45 ml PBS/0.25% FCS. The yield of recovered PBMC was 58.2±12.58%.

All cells were analyzed on either a FACScan brand flow cytometer or a FACStarPlus brand cell sorter (BDIS). The instruments were equipped with 488 nm argon lasers. The cell sorter was further equipped with a 70μ nozzle with a drop drive frequency of 27 kHz with 3 drop envelopes.

Data acquisition and analysis was performed on a Hewlett Packard 340 computer with 16 Mb of RAM using Lysis software (BDIS). Pulse width measurements were used to discriminate single cells from cell aggregates.

Prior to any experiment being run, each instrument was cleaned to assure removal of particles and debris from prior experiments. This process is described in U.S. Pat. No. 5,087,295. Briefly, a mixture of 0.07M NaOCl and NaOH in particle free deionized water were added to the sheath tank. The instrument then was run for 20 minutes by-passing the built-in 0.2μ sheath fluid filter. This tank then was emptied and particle free deionized water was added and the instrument then run for 30 minutes. The tank was again emptied and sheath fluid then was added to the tank. The fluidics system was allowed to equilibrate for 15 minutes thereafter.

Electronic stability of the FACScan instrument was checked by a continuous run for 102 hours. The instrument has a test mode which sends sequenced light pulses (approximately 2000/second) to all PMTS. A live gate was set in FL1 and FL2 in accordance with manufacturer's instructions. This gate excluded all events at or below the light pulse intensities and indicates spurious events generated by the electronics of the instrument. No spurious events were generated in the test run.

For rare event acquisition, approximately 10⁸ PBMC were run on an instrument at an acquisition rate of 5,000 to 6,000 events per second. Event acquisition was triggered on a threshold set in FL2. The threshold was set just above the upper limit of autofluorescence of the bulk of the negative cells. A strong FL2 signal on possible rare events was preferred to increase the probability of detection of rare events. It should be appreciated that fluorescence thresholds may be set for other fluorescence parameters as well in order to further refine the selection method. In addition, a live gate was set in forward and side scatter which rejects most of the very small articles such as platelets.

These procedures reduce the number of events taken to RAM and to the system's hard disc to about 1%. On average, 10⁶ events were recorded for about 10⁸ cells analyzed.

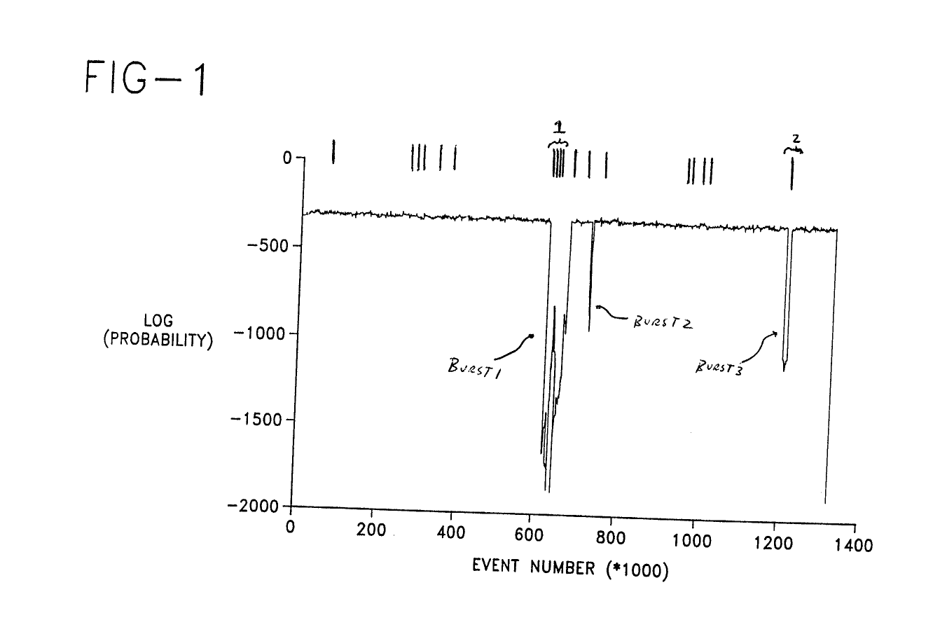

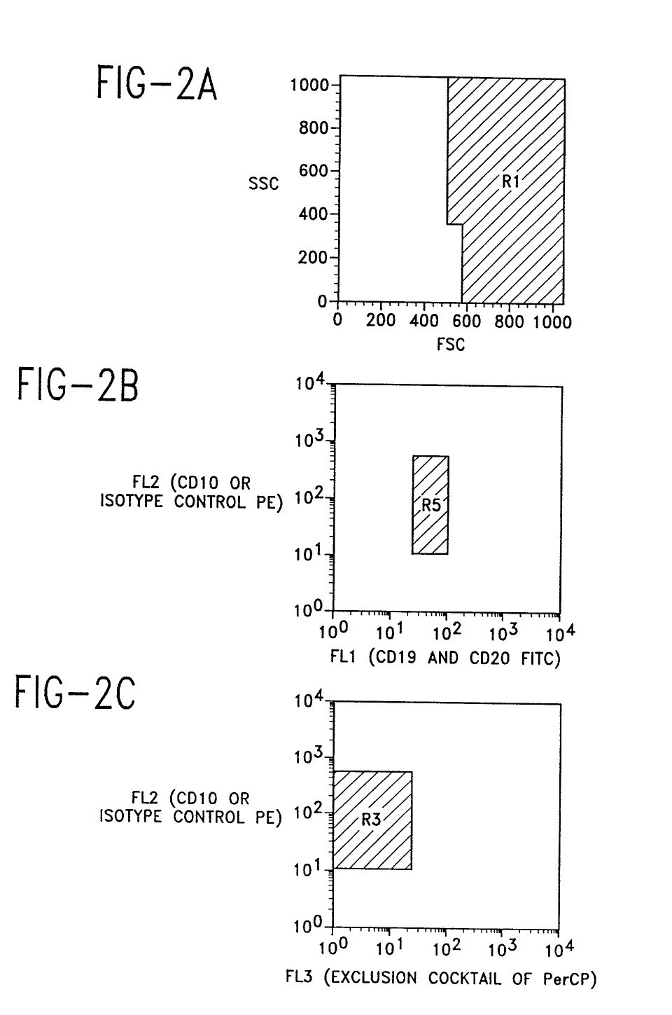

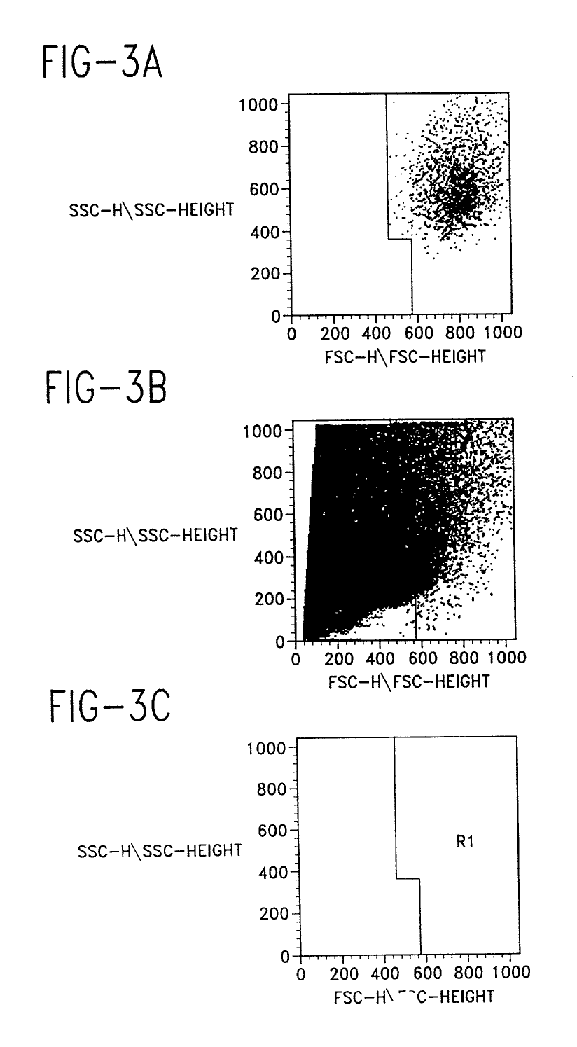

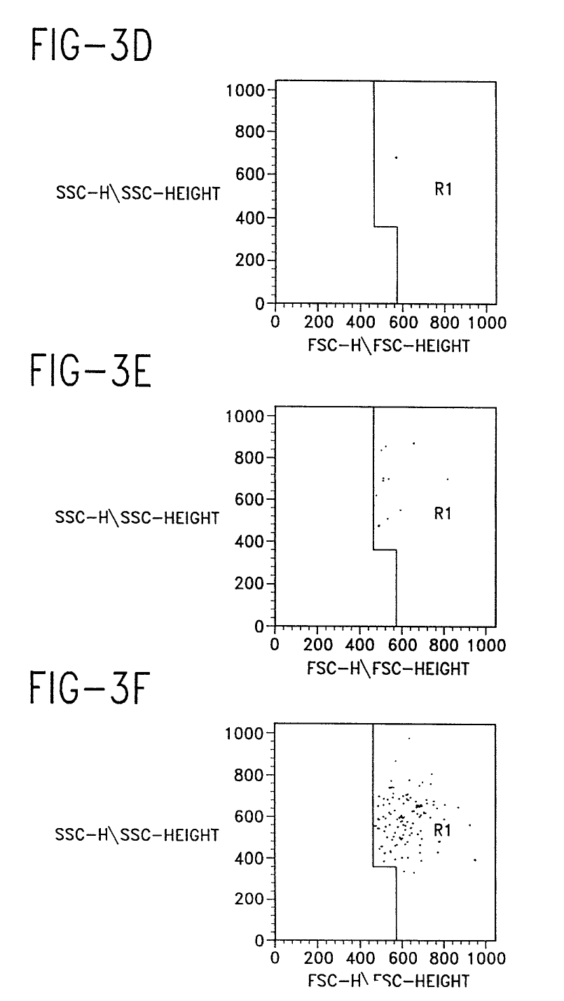

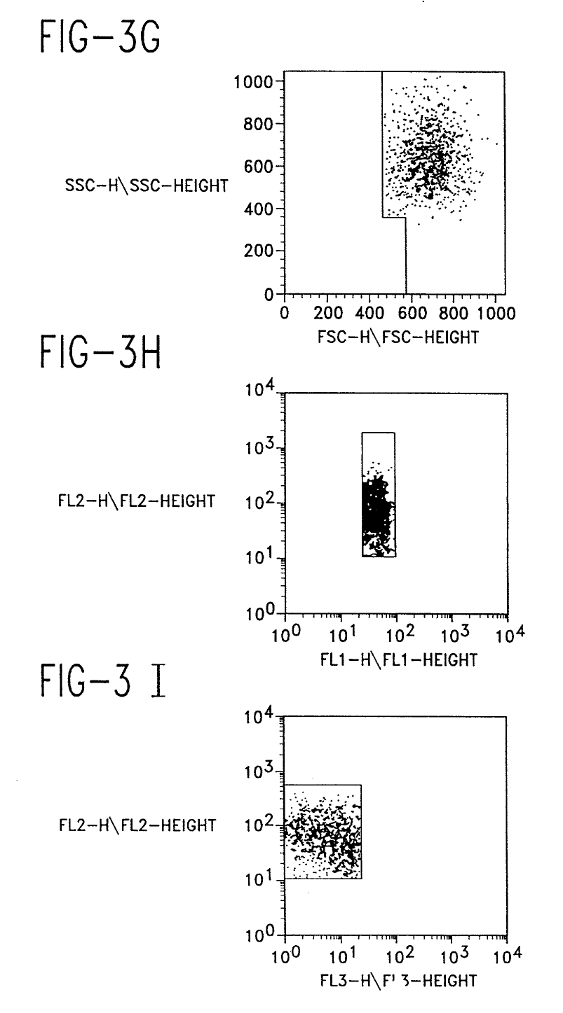

Prior to the analysis of the acquired data file, a method was applied to remove bursts of events. FIG. 1 displays event number versus the natural log of the probability of each sequence of 1000 events for a sample of 1.4×10⁶ cells containing rare cells at a frequency of 10^{-6.} The black heavy lines at the top of the figure represent rare events acquired as defined by the gates described in FIG. 2.

Three major bursts are apparent, and are identified as BURST 1, BURST 2 and BURST 3. The bursts of events were not of a Poisson distribution, and thus, represented clumps of particles or debris the inclusion of which would lead to spurious results. In two of the bursts (BURST 1 and BURST 3), “rare” events were detected, which are identified as 1 and 2 in FIG. 1. The method described below removes all events inside of a burst, and thus, excludes those “rare” events from being taken into account. The conjecture is that these events are not real rare events, or might not be, and therefore should be disregarded to assure integrity of the system. It should be readily apparent that this method has special utility for rare event detection but will have general utility to the acquisition and analysis of any dataset where fidelity is desired.

The method was applied as follows. Assume a dataset with L events consisting of N parameters digitized in B bits. Then each event Eᵢ (i−1 ... L) will receive a probability Pᵢ based on a number of W events in a window around Eᵢ. Let Hᵢⱼₖ be the number of events in the window around Eᵢ in which the jth components have a value k (i=1 ... L, j=1 ... N, k=1 ... 2^{B}). Then H^{ijk} is Poisson distributed with λjk=W^{·}Pⱼₖ where Pⱼₖ is the a priori probability that parameter j has value k. This applies only if λjk is moderate. P^{jk} can be known or estimated. In the latter case, it can be estimated during a test run or from the data itself as a ratio of the total number of events with the kth component equal to j and the total number of events L.

Any realization of Hᵢⱼₖ has a certain probability associated with it, namely:

$${P}_{ijk} = ⅇ ( - {λ}_{jk} ) \frac{{λ}_{jk}^{{H}_{ijk}}}{{H}_{ijk} !} \qquad ( 1 )$$

Multiplying the Pᵢⱼₖ over j and k gives the desired probability Pᵢ, if all Pᵢⱼₖ were independent (which they are not because the number of events in a window is limited).

However, if a fraction f of Pᵢⱼₖ with f<<W is taken, then all of the probabilities can be taken as independent when the f is spread equally over all possible j.

Once all Pᵢ have been found, a minimum filter of size W is applied. The minimum filter assigns to each event the minimum value of all the Pᵢ in the window around it. This has the effect of eliminating certain events that occur just before or after a burst.

Finally, a quantile Q of all Pᵢ is found. (Ten percent provides an appropriate measure for Q, however, Q can be set at any desired percentage.) All events with a Pᵢ less than Q are removed from the dataset, resulting in a dataset of length (1−Q)*L. It should be noted that the method will still remove Q events; however, because the probabilities for these events vary only due to statistical noise, there will be no consequence for data analysis.

In summary, the above method calculates a probability for each event (i.e., a cell) having a certain parameter j set at a certain value k. For example, one can assume that each cell or event has five parameters, with one parameter being a parameter representative of the forward light scatter (FSC), one parameter being representative of the orthogonal light scatter (SSC), and the three remaining parameters being representative of fluorescent emissions by respective markers (FL1, FL2 and FL3). The value for each parameter is the intensity of the light detected for that parameter. In other words, if the first parameter of the event (cell) is representative of the forward light scatter (FSC), the value k for that parameter is representative of the detected intensity of forward light scatter for that cell. Likewise, the respective values of k for each of the other parameters are representative of the detected light intensity for orthogonal light scatter (SSC) and the fluorescent emissions (FL1, FL2 and FL3), respectively.

The probability Pᵢ has been calculated for each event Eᵢ, based on a group or window of events surrounding the event Eᵢ, with each group or window having a predetermined number of events (e.g., 1,001 events). In this example, each group has 1,001 events; however, the method can be applied for groups or windows having any desired number of events, as long as each group or window has the same number of events.

Hence, the probability Pᵢ for each event Eᵢ is essentially determined by each of the events in the window surrounding event Eᵢ. For example, assuming that the number of events in a window is set to 1,001 (windows should have an odd number of events), then the probabilities Pᵢ can be calculated beginning at event 501 (E₅₀₁) in the total data stream of 1.4×10⁶ events (cells). In this instance, the above method arrives at an overall probability P₅₀₁ for that window given the events 1 through 1,001. That overall probability P₅₀₁ is assigned to event E₅₀₁. The above method is then applied at event 502, and thus calculates the probability P₅₀₂ of event E₅₀₂ based on the occurrence of events 2 through 1,002.

In the implementation, we use the logarithm of formula (1). Since the windows overlap, when given the Pᵢ for event 501, the probability for event 502 can be easily calculated by subtracting the contribution of event 1 and adding the contribution of event 1,002.

This calculation of the probability of each window is continued throughout the data stream to the last window (e.g., the one based on the 1.4×10⁶−500th event). Hence, since the windows overlap as described above, roughly 1.4×10⁶ probabilities (actually 1.4×10⁶−1,000 probabilities) are calculated.

Hence, each event from event 501 through event (1.4×10⁶−500) will be assigned a respective probability P₁ calculated based on its respective window of events. As can be appreciated from FIG. 1, a window having a burst or many spurious events will include many events having very low probabilities. As the multiplication (logarithmic addition) described above is carried out, the overall probability for that window will be much lower than for a window having few or no bursts or spurious events. A window having almost all normal (high probability) events but only one or a few “rare” events will not have as low a probability as a window including a burst or many spurious events. Although the probability of the rare event is very low, its multiplication (logarithmic addition) with the numerous normal (relatively high probability) events will not render the overall probability of that window as low as would a burst or a series of spurious events.

A minimum filter of size W (e.g., 1,001) is then applied to each event and reassigns to each event, the minimum value of all of the newly-assigned group probabilities within the window of events around each event. In other words, taking, for example, event 1,001, the minimum filter will consider the respective probability assigned to each of events 501 through 1501, and assign event 1,001 with the lowest of those probabilities. For event 1,002 which has been assigned a probability in the manner described above, the filter will check all the probabilities from event 502 through 1502, and reassign event 1,002 with the lowest probability of the probabilities assigned to events 502 through 1,502. This additional minimum filter processing has the effect of “expanding” a burst to take into account events immediately preceding and immediately following the burst.

The method then eliminates the lowest 10% of the probabilities. Accordingly, as shown in FIG. 1, by eliminating the groups within the lowest 10% of group probabilities, the groups including the spurious events or bursts are eliminated. Furthermore, one could use a different method to find the threshold below which events are removed (e.g., visual inspection of the calculated probabilities). Although those groups may include events falling within the definition of rare events (which are then eliminated as well), it is assumed that any such events are not true rare events, and should therefore be disregarded.

The above process can also be performed for non-overlapping windows of events. That is, for example, the window probabilities can be determined for windows of 1,000 events which do not lap (e.g., windows for events 501, 1501, 2501 and so on), and then those events are assigned with the respective calculated probabilities.

For instance, the probabilities of events 1-1,000 can be multiplied (logarithmically added) to arrive at a first window probability, the probabilities of events 1,001-2,000 can be multiplied to arrive at a second window probability and so on, until the last 1,000 events have been multiplied to arrive at the last window probability. Assuming that the 1,000 event windows are taken from a dataset having 1.4×10⁶ events, 1,400 windows probabilities will result. The calculated window probability is then assigned as the probability of each event in the window. Hence, events 1 through 1,000 will each be assigned with the probability calculated for the window of events 1 to 1,000, events 1,001 through 2,000 will each be assigned with the probability calculated for the window of events 1,001 to 2,000, and so on. Those events whose probability is within the lowest 10% of all the probabilities are then eliminated (e.g., the windows having the lowest 10% of all the probabilities are eliminated).

Alternatively, one could use a different estimate of the probability of the series of events around Eᵢ given the whole dataset, or given the data acquired so far if performed during live acquisition of data.

One could also directly estimate the probabilities that each possible overlapping or non-overlapping subset of events of a given length, and the whole dataset, are based on the same underlying probability density function. For this, one can use Kolmogorov-Smirnov statistics as described by I. T. Young, Journal of Histochemistry and Cytochemistry, pp. 935-941, the entirety of which is incorporated by reference herein.

The null hypothesis is that a subset and the whole dataset have the same underlying probability density function:

H₀: f′(x)=f(x) for all x between 0 and Xₘₐₓ

where f′(x) is the probability density function of the subset and f(x) is the probability density function of the whole dataset. With the probability density function, it is meant the probability that the given parameter for a certain event takes on the value x.

The alternate hypothesis is:

H₁: f′(x)≠f(x) for some x between 0 and Xₘₐₓ.

It is noted that f′(x) and f(x) are estimated by constructing the histograms of the parameters of the events in the subset and in the whole dataset, and then an estimate of the cumulative probability density function F′(x) and F(x) is computed by

F′(x)=sum of f′(y), with y taken from 0 to x

F (x)=sum of f(y), with y being taken from 0 to x.

The test statistic D is then computed as the maximum absolute difference between F′(x) and F(x) over all values of x:

D=max of ¦F(x)-F′(x)¦ over all x.

The null hypothesis is rejected if D>D_{crit} where D_{crit} is given in table 1 below, when n₁ and n₂ are both>15, and n₁ and n₂ are the number of events in the subset and the whole set, respectively.

TABLE 1

| confidence level | ${D}_{crit} / \sqrt{( {n}_{1} + {n}_{2} ) / ( {n}_{1} {n}_{2} )}$ |
|---|---|
| 0.10 | 1.22 |
| 0.05 | 1.36 |
| 0.01 | 1.63 |
| 0.05 | 1.73 |
| 0.001 | 1.95 |

This procedure is repeated for all possible subsets of a given length, and those subsets where the null hypothesis is rejected for any of its measured parameters are removed.

Referring to FIG. 2, as general outline of how first and second markers are used to identify rare events, such as white blood cells, is shown. First, a fluorescence threshold is applied above the autofluorescence background of the cells to exclude all events that do not have an immunofluorescence marker. Second, a region, R₁, is found in FSC and SSC that will exclude small particles, such as platelets. See FIG. 2A. It should be appreciated that if the exclusion of small particles is not necessary or may be accomplished through the use of the second markers, this step can be omitted. Finally, fluorescence regions, R₃ and R_{5,} are found for at least two combinations of the fluorescence data where rare events should occur. In this example, for pre-B cells, the plot is of the two first markers (i.e., FL2 versus FL1) which will contain only labeled B cells (see FIG. 2B), and there is a plot of one first marker versus the exclusion marker(s) FL2 versus FL3) which will contain only labeled B cells (see FIG. 2C). A rare event, thus, is defined as a cell exceeding the fluorescence threshold and falling within each of the 3 gates set.

It should be apparent to one of ordinary skill that these displays are not necessary for data analysis, but are provided as a means to visualize the steps included in the method of the invention. These steps generally are run without the need for the user to see them. It also should be apparent that as the number of first markers increases, so too will the number of gates set for the various combination of first markers.

The following examples described in more detail the nature of this invention.

### Example I

250, 2,500 and 25,000 pre-B cells were sorted into 2.5×10⁸ PBMC. This represents a concentration of pre-B cells of 10⁻⁶, 10⁻⁵ and 10⁻⁴ respectively. Sorting was done using a FACStar^{Plus} cell sorter using the instrument's “count mode” and “pulse processor.” Count mode delivers a pre-set number of cells with pre-set characteristics. The pulse processor measures pulse width which is used to discriminates single cells from aggregates.

Prior to Sorting, sort accuracy was checked as follows. An aliquot of the pre-B cells was labeled with nuclear UV excitable dye Hoechst 33 342 (10 μg/ml) for 10 minutes at 37° C. for examination of cells under a fluorescence microscope. After washing and suspending the pre-B cells in PBS, a defined number of cells were sorted using a FACStarPlu5 based upon forward and side scatter gates to exclude dead cells which have lower forward and side scatter. The number of cells sorted was verified under the fluorescence microscope. Sorting efficiency was determined to be greater than 97%.

### Example II

250 pre-B cells were sorted into 2.5×108 PBMC. The pre-B cells were stained with Hoechst 33 342 as in Example 1 and with monoclonal antibodies as described above. Sequential aliquots of cells were taken out of the staining solution just prior to analysis. The cells were washed twice, resuspended in PBS and were immediately analyzed on a FACStar^{Plus} cells sorter which had been cleaned as described above. Cells were sorted onto a microscope slide if they were FITC⁺(i.e., FL1⁺), PE⁺(i.e., FL2+) and PerCP (i.e., FL3). Cells were sorted onto a new slide after every 106 cells analyzed. Slides were examined under a UV fluorescence microscope. The presence of pre-B cells was verified on the slide by UV excitation. This experiment confirmed that the sort strategy worked.

### Example III

0, 250, 2,500 and 25,000 pre-B cells were sorted into 2.5×10₈ PBMC. This represents a frequency of pre-B cells of 10⁻⁶, 10⁻⁵ and 10⁻⁴ respectively. The pre-B cells were stained as in Example I with the first and second markers described above. The cells were washed twice, resuspended in PBS and were immediately analyzed on a FACStarPlus cells sorter which had been cleaned as described above.

Referring to FIGS. 3H and I respectively, gates were established in a plot of FL2 versus FLI and FL3 versus FL1 for pre-B cells at a concentration of one in 10⁻⁴ PBMC. The gates, referred to as “RS” in H and “R3” in 1, were set so as to include the most likely areas were pre-B cells should occur.

The −R5 and R3 gates then were applied to the list mode data from the analysis, and a plot of SSC versus FSC was constructed. Referring to FIG. 3A, the gate “RI” was constructed in the scatter plot so as to include all of the pre-B cells. All three gates then were applied to various aliquots.

Referring to FIGS. 3B and C respectively, PBMC were analyzed using anti-KLH as the marker without and with the R1, R3 and R5 gates. As can be seen, for ungated data numerous events are displayed in a plot of SSC versus FSC; however, when the gates are applied, only one event in 10⁶ events analyzed is recorded. Referring to FIG. 3D, when 0 pre-B cells are seeded into PBMC and 10⁶ events are analyzed, only one event in 10⁶ appears as background.

Referring to FIGS. 3E, F and G respectively, gates RI, R3 and R5 were applied to listmode data recorded from the analysis of 10⁻⁶, 10⁻⁵ and 10⁻⁴ pre-B cells seeded into at least 10⁶ PBMC. For 10⁻⁶ seeded cells, 12-17 events were recovered in 4 replicate experiments. For 10⁻⁵ seeded cells, 45-136 pre-B cells were recovered in 3 replicate experiments. For 10⁻⁴ seeded cells, 371-1291 pre-B cells were recovered in 3 replicate experiments. As expected, the number of recovered cells increases by a factor of 10 as the number of seeded cells increases by a factor of 10.

All publications and patents mentioned in this specification are indicative of the level of ordinary skill in the art to which this invention pertains. All publications and patents are herein incorporated by reference to the same extent as if each individual publication or patent was specifically and individually indicated to be incorporated by reference.

What is claimed is:

1. A method for eliminating spurious events from an n-dimensional datastream of events produced by flow cytometry, the method comprising the steps of:
calculating the probability of occurrence for each event in the datastream;
calculating, based on the probabilities calculated in the event probability calculating step, the probability of occurrence for each of a plurality of subsets of events of the datastream, each subset comprising a plurality of consecutive events; and
eliminating those of said subsets having a probability of occurrence below a predetermined threshold probability.

2. A method as claimed in claim 1, wherein:
each of the subsets includes events which are included in at least one other of the subsets.

3. A method as claimed in claim 1, wherein:
each of the subsets includes events different from any of the events in any other of the subsets.

4. A method as claimed in claim 1, further comprising the step of:
analyzing those of said events remaining after the eliminating step to detect the presence of events having a predetermined probability.

5. A method as claimed in claim 4, wherein:
each event includes parameters representative of characteristics of a cell identifiable by flow cytometry; and
the analyzing step analyzes the remaining events to detect the presence of events representative of cells having at least one particular characteristic.

6. A method as claimed in claim 1, wherein:
the subset probability calculating step calculates the probability of occurrence for said each of the plurality of subsets in accordance with a Poisson distribution.

7. A method as claimed in claim 1, wherein:
the event probability calculating step calculates the probability of occurrence for each event based upon all of the events in the datastream.

8. A method as claimed in claim 1, wherein:
the event probability calculating step calculates the probability of occurrence of each event based upon events in a test datastream.

9. A method for eliminating spurious events from an n-dimensional datastream of events produced by flow cytometry, the method comprising the steps of:
dividing the datastream of events into subsets, each of which comprises a plurality of consecutive events which each have a parameter;
generating, for each subset, a histogram of the parameters of the events in the subset;
generating a histogram of the parameters of the events in the entire datastream;
comparing the histograms of each of the subsets to the histogram of the entire datastream to determine which of the histograms of the subsets differ by a predetermined amount from the histogram of the entire datastream; and
eliminating those subsets whose histogram is determined to differ from the histogram of the entire datastream by the predetermined amount.

10. A method as claimed in claim 9, wherein:
each of the subsets includes events which are included in at least one other of the subsets.

11. A method as claimed in claim 9, wherein:
each of the subsets includes events different from any of the events in any other of the subsets.

12. A method as claimed in claim 9, further comprising the step of:
analyzing those of said events remaining after the eliminating step to detect the presence of events having a predetermined probability.

13. A method as claimed in claim 12, wherein:
each event includes parameters representative of characteristics of a cell identifiable by flow cytometry; and
the analyzing step analyzes the remaining events to detect the presence of events representative of cells having at least one particular characteristic.

14. A method as claimed in claim 9, wherein:
the two histogram generating steps uses Kolmogorov-Smirnov statistics to generate the histograms for each of the subsets and the histogram for the entire datastream.
